(19) United States
(12) Patent Application Publication
(10) Pub. No.: US 2014/0296096 A1
(43) Pub. Date: Oct. 2, 2014

(54) PROSTATE CANCER MARKERS AND USES THEREOF

(71) Applicant: Oslo universitetssykehus HF, Oslo (NO)

(72) Inventors: Alicia Llorente, Oslo (NO); Tore Skotland, Nittedal (NO); Kirsten Sandvig, Nittedal (NO)

(73) Assignee: Oslo universitetssykehus HF, Oslo (NO)

(21) Appl. No.: 14/226,912

(22) Filed: Mar. 27, 2014

Related U.S. Application Data
(60) Provisional application No. 61/805,648, filed on Mar. 27, 2013.

(51) Int. Cl. G01N 33/574

(52) U.S. Cl. CPC G01N 33/57434
USPC 506/9; 435/7.92; 435/7.94; 435/7.9; 435/7.23; 506/18; 435/23

(57) ABSTRACT

The present invention relates to compositions and methods for cancer diagnosis, research and therapy, including but not limited to, cancer markers. In particular, the present invention relates to polypeptides found in extracellular microvesicles as diagnostic and screening markers, and clinical targets for cancer (e.g., prostate cancer).

### CROSS-REFERENCE TO RELATED APPLICATIONS

The present application claims priority to pending U.S. Provisional Patent Application No. 61/805,648, filed Mar. 27, 2013, the contents of which are incorporated by reference in its entirety.

### FIELD OF THE INVENTION

The present invention relates to compositions and methods for cancer diagnosis, research and therapy, including but not limited to, cancer markers. In particular, the present invention relates to polypeptides found in extracellular microvesicles as diagnostic and screening markers, and clinical targets for cancer (e.g., prostate cancer).

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed.

### SUMMARY OF THE INVENTION

The present invention relates to compositions and methods for cancer diagnosis, research and therapy, including but not limited to, cancer markers. In particular, the present invention relates to polypeptides found in extracellular microvesicles as diagnostic and screening markers, and clinical targets for cancer (e.g., prostate cancer).

Embodiments of the present invention provide compositions, kits, uses, and methods useful in the detection and screening of prostate cancer. For example, in some embodiments, the present invention provides uses and methods of identifying prostate cancer in a sample from a subject, comprising: detecting the presence, absence, or altered level (e.g. increased or decreased level) of at least one polypeptide selected from those described in Tables 1-5 and FIG. 5 herein (e.g., CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, or mucin-5B) with a reagent that specifically detects the polypeptide in a biological sample from a subject (e.g., a sample comprising a microvesicle); and identifying the presence of prostate cancer in the sample when the at least one polypeptide is present in the sample. In some embodiments, the method further comprises the step of isolating a microvesicle from the biological sample. In some embodiments, detection is carried out utilizing a method selected from, for example, a spectrometry technique, a chromatography technique, or an immunoassay. In some embodiments, the reagent is an antibody (e.g., monoclonal or polyclonal antibody) that specifically binds to the polypeptide. In some embodiments, the microvesicle is released from a epithelial cell (e.g., a prostate cancer cell (e.g., metastatic or localized prostate cancer cell).

The present invention is not limited to a particular sample. Examples include, but are not limited to, tissue, blood, plasma, serum, urine, urine supernatant, urine cell pellet, semen, prostatic secretions or prostate cells.

In some embodiments, the method further comprises the step of enriching the sample for the presence of extracellular vesicles (e.g., by isolating vesicles). The present invention is not limited to a particular method of isolating microvesicles. For example, in some embodiments, microvesicles are isolated from the sample by antibody capture (e.g., a bead comprising an antibody that specifically binds to the microvesicle). In some embodiments, the antibody specifically binds to epithelial cell adhesion molecule (EpCAM). In some embodiments, the detecting further comprises detecting the presence of a complex of the polypeptide and the reagent. In some embodiments, the method further comprises the step of treating the subject for prostate cancer when the polypeptide is detected.

In further embodiments, the present invention provides a kit, comprising reagents for detection of at least one (e.g., two, 3, 4, or all 5) polypeptide selected from CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, or mucin-5B. In some embodiments, the reagent is an antibody that specifically binds to the polypeptide.

Additional embodiments provide a complex, comprising: at least 1 (e.g., two, 3, 4, or all 5) polypeptide selected from CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, or mucin-5B, each of which is complexed to a reagent that specifically detects (e.g., binds) the polypeptide. In some embodiments, the reagent is an antibody that specifically binds to the polypeptide.

In further embodiments, the present invention provides the use of a reagent that specifically detects the presence of at least one polypeptide selected from those disclosed in Tables 1-4 and FIG. 5 herein (e.g., CUB domain-containing protein 1 protein (CDCP1), CD151, CD147, translationally controlled tumor protein (TCTP) or neuropilin) in a microvesicle isolated from a biological sample in the identification of prostate cancer in a subject.

Additional embodiments are described herein.

### DESCRIPTION OF THE FIGURES

FIG. 1 shows microvesicles released by PC-3 cells. A. Microvesicles were isolated from PC-3 cells after 1, 2, and 3 days. B. Microvesicles were isolated after 3 days from conditioned media of PC-3 cells. C. Venn diagram of the proteins identified in two independent proteomic analysis of PC-3 microvesicles.

FIG. 2 shows classification of proteins in PC-3 cell-released microvesicles by GOslim annotations. Proteins identified in PC-3 cell-derived microvesicles were classified by their cellular location (A) (er: endoplasmic reticulum) and biological processes (B) using Genome Ontology Slim annotations.

FIG. 3 shows analysis of CDCP1 as a prostate cancer biomarker. The amounts of protein loaded per lane are indicated in the figure. CDCP1 (A) and calreticulin (B) were then detected in lysates (Lys) and microvesicles (Ves) by Western blot. C. Microvesicles were isolated from conditioned media of PC-3 cells and LnCAP. D. Microvesicles were isolated from the conditioned media of PC-3 cells and immunoisolated by EpCAM-dynabeads.

FIG. 4 shows analysis of CD151 and CD147 as prostate cancer biomarkers. Microvesicles were isolated from PC-3 and RWPE-1 cells. CD151 (A) and CD147 (B) were then detected in lysates (Lys) and microvesicles (Ves) by Western blot.

FIG. 5 shows proteins found in microvesicles released by the prostate cancer cell line PC-3.

### DEFINITIONS

To facilitate an understanding of the present invention, a number of terms and phrases are defined below:

As used herein, the term “microvesicle” refers to a membrane-enclosed sac released from a cell. In some embodiments, microvesicles are released by direct budding from the plasma membrane or/and fusion of multivesicular bodies (MVB) with the plasma membrane. In some embodiments, the terms “exosome,” “prostasome,” “ectosome,” “exosome-like vesicle,” “shedding vesicle” and “membrane particle” are used interchangeably with “microvesicle.”

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or presence or absence or mutation status in cancer markers described herein indicative of cancer is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing prostate tissue in a subject” refers to the identification of one or more properties of a prostate tissue sample (e.g., including but not limited to, the presence of cancerous tissue, the presence or absence or mutation status of cancer markers, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for cancer diagnosis, research and therapy, including but not limited to, cancer markers. In particular, the present invention relates to polypeptides found in extracellular microvesicles as diagnostic and screening markers, and clinical targets for cancer (e.g., prostate cancer).

It has been shown that a number of different cell types release microvesicles to the extracellular environment (Johnstone, et al., (1989) Blood. 74, 1844-1851; Thery, et al., (1999) J. Cell Biol. 147, 599-610; Conde-Vancells, et al., (2008) J. Proteome. Res. 7, 5157-5166; Llorente, et al., (2004) J. Cell Sci. 117, 5343-5351; Mathivanan, et al., (2010) Mol. Cell. Proteomics. 9, 197-208). Cancer cells release more microvesicles that control cells. It has for example been shown that plasma samples of patients with advanced lung cancer, ovarian cancer and prostate cancer had higher levels of microvesicles compared to control patients (Rabinowits, et al., (2009) Clin. Lung Cancer. 10, 42-46; Taylor, et al., (2008) Gynecol. Oncol. 110, 13-21; Tavoosidana, et al., (2011) Proc. Natl. Acad. Sci. U.S. A 108, 8809-8814). Microvesicles are able to affect neighboring cells in various ways, for example by inducing intracellular signaling or by transferring different molecules such as proteins, mRNAs or microRNAs to cells (Valadi, et al., (2007) Nat. Cell Biol. 9, 654-659; Babiker, et al., (2002) Am. J. Reprod. Immunol. 47, 183-192). Concerning cancer, microvesicles have been proposed to contribute to cancer cell survival, invasiveness and metastases (van Doormaal, et al., (2009) Neth. J. Med. 67, 266-273; Al-Nedawi, et al., (2009) Cell Cycle. 8, 2014-2018). Furthermore, microvesicles are a source of cancer biomarkers since they carry tumor-related molecules (Al-Nedawi, et al., (2009) Cell Cycle. 8, 2014-2018; Nilsson, et al., (2009) Br. J. Cancer. 100, 1603-1607; Simpson, et al., (2009) Expert. Rev. Proteomics. 6, 267-283). Promising results show that plasma samples from ovarian cancer patients contain claudin-4-containing exosomes (Li, et al., (2009) BMC. Cancer. 9:244,) and that plasma from melanoma patients contain high levels of microvesicles expressing CD63 and caveolin-1 (Logozzi, et al., (2009) PLoS. One. 4, e5219).

In addition to blood plasma, microvesicles have been found in many biological fluids such as urine, seminal fluid, saliva, tear fluid, breast milk and amniotic fluid. However, body fluids contain a mixture of microvesicles originating from different cells types that makes difficult the analysis and interpretation of proteomic studies. Therefore, several groups have performed proteomic studies on cell lines originating from specific cancer diseases (Mathivanan, et al., (2010) Mol. Cell. Proteomics. 9, 197-208; Welton, et al., (2010) Mol. Cell. Proteomics. 9, 1324-1338; Mears, et al., (2004) Proteomics. 4, 4019-4031; Choi, et al., (2007) J. Proteome. Res. 6, 4646-4655).

There has recently been a considerable increase in the number of articles published on microvesicles (for reviews see (Al-Nedawi, et al., (2009) Cell Cycle. 8, 2014-2018; Simpson, et al., (2009) Expert. Rev. Proteomics. 6, 267-283; Cocucci, et al., (2009) Trends Cell Biol. 19, 43-51; Thery, et al., (2009) Nat. Rev. Immunol. 9, 581-593; Pilzer, et al., (2005) Springer Semin. Immunopathol. 27, 375-387; Simons, et al., (2009) Curr. Opin. Cell Biol. 21, 575-581; Bobrie, et al., (2011) Traffic. 12, 1659-1668). Different names (microvesicles, exosomes, prostasomes, ectosomes, exosome-like vesicles, membrane particles), isolation protocols and vesicle sizes can be found in the literature based on the source of microvesicles and/or release mechanism. Scientists working in this field are trying to get consensus on these issues. Even though there are still many unanswered questions, microvesicles seem to be released by two main mechanisms: direct budding from the plasma membrane Trends Cell Biol. 19, 43-51) and fusion of multivesicular bodies (MVB) with the plasma membrane, a process that leads to the release of the internal vesicles contained in the MVB (Simons et al., supra). Microvesicles that originate from the plasma membrane are often referred as shedding vesicles. These vesicles are 100-1000 nm in diameter, sediment at 10,000 g (Thery et al., supra) and are often secreted when cells are submitted to stress conditions. However, microvesicles that originate from MVB, exosomes, typically have a size diameter of 50-100 nm and sediment at 100,000 g (Thery et al., supra). Cells may contain different types of MVBs, and that there may be a specific MVB population given rise to exosomes (Simons et al., supra).

Experiments conducted during the course of development of embodiments of the present invention studied microvesicles from the metatatic prostate cancer cell line PC-3. The microvesicles pelleted at 100,000 g from the culture medium of these cells have previously been referred as prostasomes (Llorente, et al., (2004) J. Cell Sci. 117, 5343-5351; Llorente, et al., (2007) Eur. J. Cell Biol. 86, 405-415), a term used to name vesicles released by prostate cells (Ronquist, G., and Brody, I. (1985) Biochim. Biophys. Acta. 822, 203-218; Brody, et al., (1983) Ups. J. Med. Sci. 88, 63-80). There is strong evidence that most microvesicles released from PC-3 cells are secreted in a similar way as exosomes (Llorente, et al., (2004) J. Cell Sci. 117, 5343-5351; Llorente, et al., (2007) Eur. J. Cell Biol. 86, 405-415).

Cancer biomarkers are invaluable tools for cancer detection, prognosis and treatment. Recently, microvesicles have appeared as a novel source for cancer biomarkers. Experiments conducted herein describe a proteomic analysis of microvesicles released to the extracellular environment by the metastatic prostate cancer cell line PC-3. Using nanocapillary liquid chromatography-tandem mass spectrometry 266 proteins were identified with 2 or more peptide sequences. Further analysis showed that 16% of the proteins were classified as extracellular and that intracellular proteins were annotated in a variety of locations. Concerning biological processes, the proteins found in PC-3 cell-released microvesicles are mainly involved in transport, cell organization and biogenesis, metabolic process, response to stimulus and regulation of biological processes. Several of the proteins identified (tetraspanins, annexins, Rab proteins, integrins, heat shock proteins, cytoskeletal proteins, 14-3-3 proteins) have previously been found in microvesicles isolated from other sources. However, some of the proteins are specific to the vesicular population released by the metastatic prostate cancer PC-3 cell line. Among these proteins are the tetraspanin protein CD 151 and the glycoprotein CUB domain-containing protein 1. The results show these proteins find use as biomarkers for prostate cancer.

### I. Diagnostic and Screening Methods

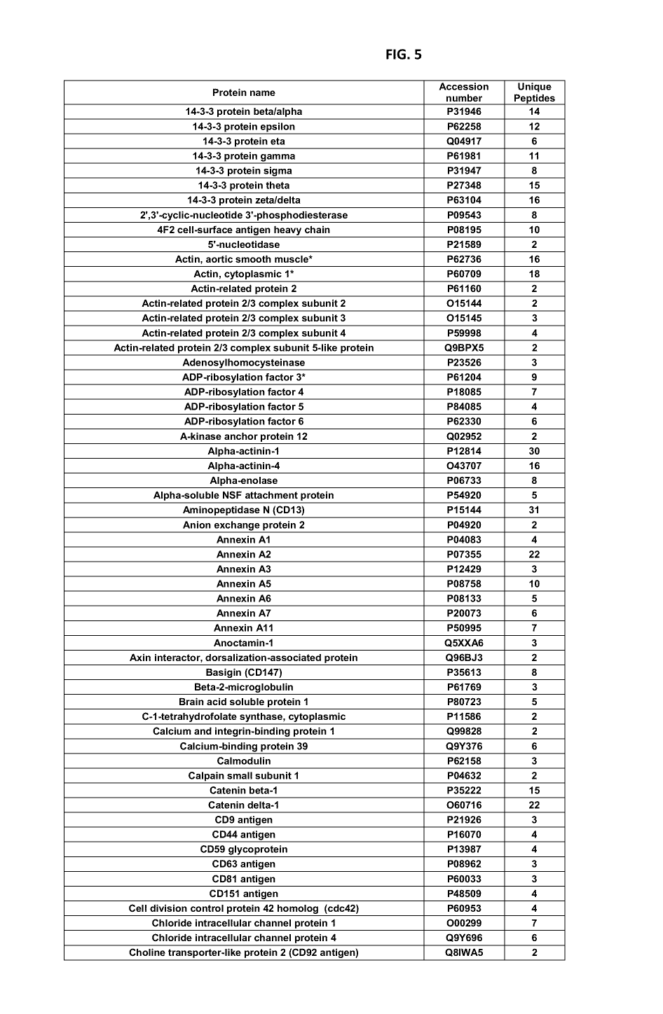

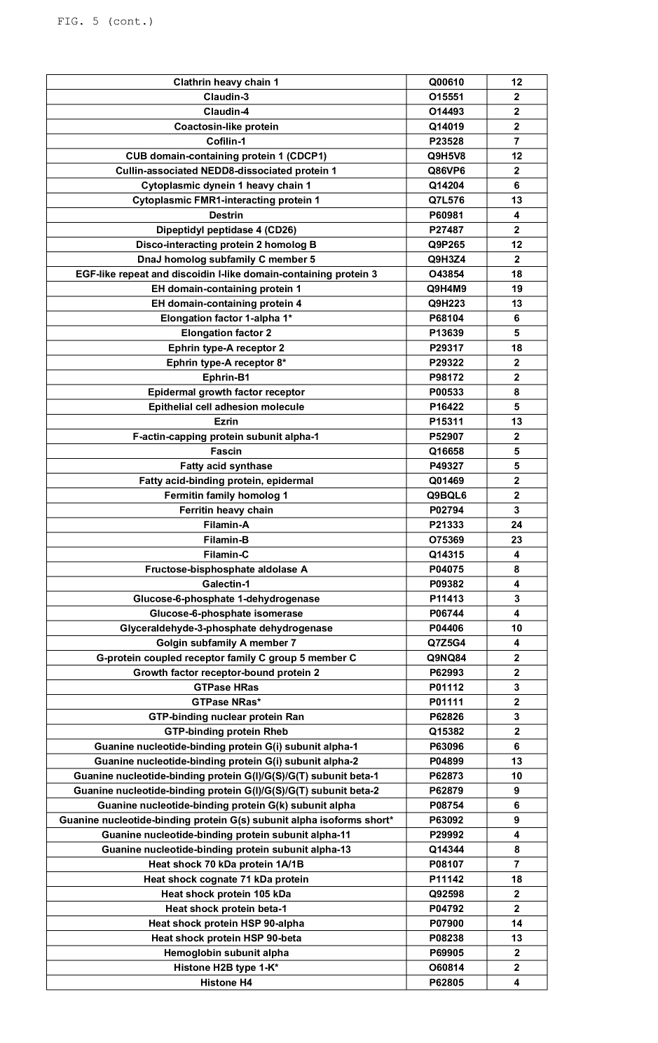

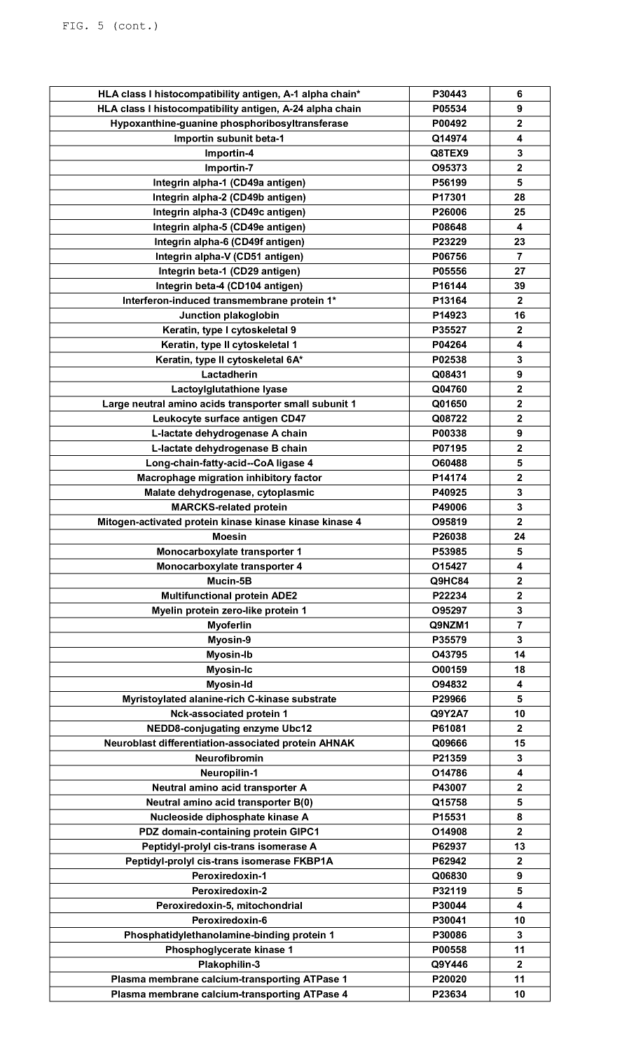

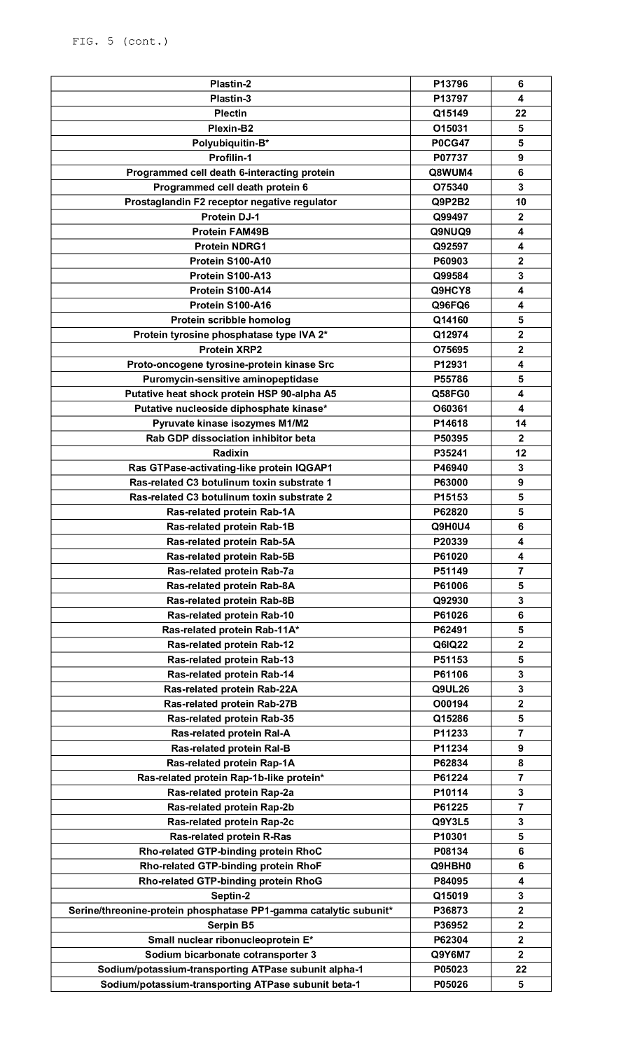

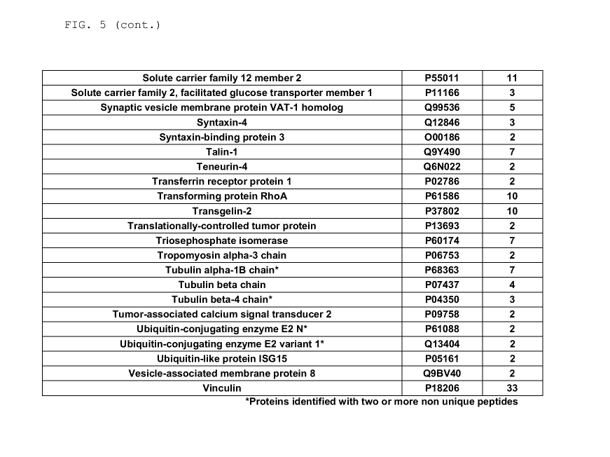

As described herein, embodiments of the present invention provides compositions, kits, systems, uses, and methods for identifying and characterizing prostate cancer. In some embodiments, the compositions, kits, systems, uses, and methods comprise detecting polypeptides found in microvesicles derived from prostate cancer cells but not microvesicles from non-prostate cancer cells or polypeptides with altered (e.g., increased or decreased) levels of expression in microvesicles from prostate cancer cells relative to non-prostate cancer cells. In some embodiments, the prostate cancer microvesicle specific polypeptides comprises one or more (e.g., 2, 3, 4, 5, 6, 7, 8, 9, 10, 15, 20 or more) of those described in Tables 1-5 and FIG. 5 herein (e.g., including but not limited to, CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, or mucin-5B).

The present invention is not limited to a particular method of detecting polypeptides in microvesicles. In some embodiments, samples are enriched for microvesicles prior to detection of the polypeptides. In some embodiments, microvesicles are first isolated from a biological sample (e.g., blood, plasma, serum, urine, prostate secretions, prostate cells, and the like). The present invention is not limited to a particular method of isolating microvesicles. In some embodiments, detection methods specifically isolate microvesicles derived from prostate cells (e.g., epithelial cells). For example, in some embodiments, microvesicles from epithelial cells are isolated using antibody capture with an antibody that specifically binds to epithelial cells (e.g., epithelial cell adhesion molecule (EpCAM)). In some embodiments, antibodies are bound to a bead or particle that facilitates isolation of microvesicles.

In other embodiments, polypeptides are detected using a suitable technique such as those described below or herein (e.g., chromatograph, spectroscopy, or immunological techniques).

In some embodiments, immunoassays are utilized to detect polypeptides. In some embodiments, proteins are detected by their binding to an antibody raised against the protein. The generation of antibodies is described below.

Antibody binding is detected by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference

In some embodiments, microarrays including, but not limited to: protein microarrays, and antibody microarrays are utilized to detect microvesicle polypeptides. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a cancer marker) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

In some embodiments, the present disclosure provides methods of determining a treatment course of action based on results of diagnostic assays described herein. For example, in some embodiments, subjects found to have one or more markers indicative of a diagnosis of prostate cancer are administered a treatment for prostate cancer (e.g., chemotherapy, surgery, radiation, and the like). In some embodiments, subjects undergoing treatment are monitored using the diagnostic assays described herein. In some embodiments, subjects are screened for the presence of the cancer markers at multiple times (e.g., daily, weekly, monthly, annually, or less often) before, during, or after treatment for prostate cancer. In some embodiments, the results are used to alter (e.g., start, stop, or change) treatment for prostate cancer.

Embodiments of the present invention provide kits and compositions for detecting the presence of prostate cancer microvesicle polypeptides. In some embodiments, kits provide one or more reagents useful, necessary, or sufficient for detection of prostate cancer microvesicle polypeptides. Examples include, but are not limited to, antibodies, reagents, controls, and the like. The antibody compositions may also be provided in the form of an array.

In some embodiments, the present disclosure provides complexes comprising one or more (e.g., 2, 3, 4, or all 5) of cancer markers disclosed herein (e.g., CUB domain-containing protein 1 protein (CDCP1), CD151, CD147, translationally controlled tumor protein (TCTP), or neuropilin) complexed to a reagent (e.g., antibody) that specifically detects the cancer marker.

### II. Antibodies

The present invention provides isolated antibodies. In preferred embodiments, the present invention provides monoclonal antibodies that specifically bind to an isolated polypeptide comprised of at least five amino acid residues of the cancer markers described herein (e.g., CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, or mucin-5B). These antibodies find use in the diagnostic and screening methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody producing cells, an individual animal whose antibody titer has been confirmed (e.g., a mouse) is selected, and 2 days to 5 days after the final immunization, its spleen or lymph node is harvested and antibody producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS 1, P3U1, SP2/0, AP 1 and the like. The proportion of the number of antibody producer cells (spleen cells) and the number of myeloma cells to be used is preferably about 1:1 to about 20:1. PEG (preferably PEG 1000 PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti immunoglobulin antibody (if mouse cells are used in cell fusion, anti mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM 101, Nissui Seiyaku) and the like can be used. Normally, the cultivation is carried out at 20° C. to 40° C., preferably 37° C. for about 5 days to 3 weeks, preferably 1 week to 2 weeks under about 5% CO2 gas. The antibody titer of the supernatant of a hybridoma culture can be measured according to the same manner as described above with respect to the antibody titer of the anti protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention can be used as the immunogen. Further, fragments of the protein may be used. Fragments may be obtained by any methods including, but not limited to expressing a fragment of the gene, enzymatic processing of the protein, chemical synthesis, and the like.

### III. Drug Screening Applications

In some embodiments, the present invention provides drug screening assays (e.g., to screen for anticancer drugs). The screening methods of the present invention utilize cancer markers described herein. For example, in some embodiments, the present invention provides methods of screening for compounds that alter (e.g., increase or decrease) the expression or activity of cancer markers described herein. The compounds or agents may interfere with transcription, by interacting, for example, with the promoter region. The compounds or agents may interfere with mRNA (e.g., by RNA interference, antisense technologies, etc.). The compounds or agents may interfere with pathways that are upstream or downstream of the biological activity of cancer markers. In some embodiments, candidate compounds are antisense or interfering RNA agents (e.g., oligonucleotides) directed against cancer markers. In other embodiments, candidate compounds are antibodies or small molecules that specifically bind to a cancer markers regulator or expression products and inhibit its biological function.

In one screening method, candidate compounds are evaluated for their ability to alter cancer marker expression or activity by contacting a compound with a cell expressing a cancer marker and then assaying for the effect of the candidate compounds on expression or activity.

### EXPERIMENTAL

The following examples are provided in order to demonstrate and further illustrate certain preferred embodiments and aspects of the present invention and are not to be construed as limiting the scope thereof.

### Example 1

### A. Methods

### Materials

Dithiothreitol (GE Healthcare, Oslo, Norway), iodoacetamide (Sigma-Aldrich Norway), trypsin porcine from (Promega, Madison, USA), nC₈ Empore 3M Extraction Disks (Agilent Technologies, Palo Alto, USA), antibody to caveolin-1 (BD Biosciences, San Diego, Calif., USA), antibody to CUB domain-containing protein 1 (CDCP1) (R&DSystems, Abingdon, UK), antibodies to CD147 and CD151 (Abcam, Cambridge, UK), antibody to calreticulin (Stressgen, Enzo Life Sciences), antibody to MOC31 (anti-EpCAM) (IQ Products, Groningen, The Netherlands). DMEM/F-12 (1:1 Mix of DMEM and Ham's F-12) medium, RPMI 1640 medium and keratinocyte-serum free medium kit with L-glutamine, epidermal growth factor and bovine pituitary extract were from Gibco, (Invitrogen Dynal, Norway). The Immunomagnetic M450 Dynabeads (diameter 4.5 μm) were from Invitrogen (Oslo, Norway). Bicinchoninic acid protein assay kit and Western blotting detection reagents were from Pierce (Rockford, Ill., USA).

### Cell Culture

The epithelial human prostate cancer cell line PC-3 (Kaighn, et al., (1979) Invest Urol. 17, 16-23) obtained from the American Type Culture Collection was maintained in a 1:1 mixture of Ham's F12 medium and Dulbecco's modified Eagle's medium supplemented with 7% foetal calf serum, 100 units/ml penicillin and 100 μg/ml streptomycin. The epithelial human prostate cell line RWPE-1 was obtained from the American Type Culture Collection and grown in keratinocyte serum-free medium supplemented with bovine pituitary extract (0.05 mg/ml) and EGF (5 ng/ml), 100 units/ml penicillin, and 100 μg/ml streptomycin. The nonmetastatic prostate cancer cell line LNCaP was grown in RPMI medium supplemented with 10% foetal calf serum, 100 units/ml penicillin and 100 μg/ml streptomycin. Cells were maintained at 37° C. in an atmosphere of 5% CO₂/95% air.

### Microvesicle Isolation

PC-3 cells were grown in serum-free cell culture medium for 3 days and microvesicles were isolated from the culture medium as previously described (Llorente, et al., (2004) J. Cell Sci. 117, 5343-5351). Serum-free medium was used to avoid contamination with vesicles contained in serum. The culture medium was centrifuged to remove cell debris first at 400 g for 10 mM and then at 10,000 g for 30 mM Vesicles were then collected by ultracentrifugation at 100,000 g for 2 h in a SW40 or SW28 rotor, washed with a large volume of phosphate-buffered saline, and then concentrated by ultracentrifugation at 100,000 g for 2 h in a SW40 rotor first, and then in a TLA 120.1 rotor. For mass spectrometry analysis microvesicles were isolated from 5-7×10⁷ cells.

### Protein Determination

PC-3 cells and microvesicles were lysed in lysis buffer (25 mM Tris-HCl, 125 mM NaCl, 5 mM EDTA, 1% Triton X-100, SDS 0.1%, deoxycholate 2 g/l, pH 7.4) in the presence of a protease inhibitor mixture. Then, the protein content was determined using a bicinchoninic acid protein assay kit according to the manufacturer's instructions.

### SDS-PAGE

Pelleted microvesicles pellets directly solubilised in loading buffer. Whole cell lysates were solubilised in lysis buffer (25 mM Tris-HCl, 125 mM NaCl, 5 mM EDTA, 1% Triton X-100, SDS 0.1%, deoxycholate 2 g/l, pH 7.4) in the presence of a protease inhibitor mixture. Sample buffer was added to the cell lysates after removal of insoluble material. Microvesicles and lysate samples were then subjected to SDS-PAGE. Microvesicle samples destined for LC-MSMS were run in 4-20% gradient gels, stained with Coomassie Blue and cut in pieces.

### Peptide Generation

For generation of peptides, microvesicle proteins contained in gel pieces were treated as followed. First, possible disulfide bridges between cysteines were broken by reduction with dithiothreitol and alkylated with iodoacetamide to prevent oxidation and formation of new disulfide bridges. Proteins were then digested in-gel by trypsin for 16 hours at 37° C. The protease activity was stopped by acidification using 2% formic acid. Samples were then filtered in a homemade nC8 StageTip (Stop and go extraction Tip, using nC₈ Empore 3M Extraction Disks column (Rappsilber, Ishihama et al. 2003). The sample was eluted through the column followed by 20 μl 60% acetonitrile/0.1% formic acid. Acetonitrile was removed by evaporation and the sample volume was reduced to about 10 μl before further analysis.

### Nano LC-MS/MS Analysis

Peptides from digested samples were first separated on an ultimate 3000 nano-LC (Dionex Corporation, USA), equipped with a nC₁₈ enrichment column (C₁₈ Pepmap 100 from Dionex, 5 μm particle size, 100 Å pore size, 300 μm i.d.×5 mm) and an nC₁₈ analytical column (C₁₈ Pepmap 100 from Dionex; 3 μm particle size, 100 Å pore size, 75 μm×150 mm) About 10 μl of each sample (concentrated to 1-2 μl and diluted to about 20 μl with 0.1% FA/2% CAN) was injected. Flow rate enrichment column 25 μl/min Flow rate analytical column 300 nl/min. The eluting peptides from the nano-LC were then ionized (ESI, ElectroSpray Ionization) (capillary voltage 2700 V, cone voltage 100 V) and analyzed with a QToF Global using automatic MS to MSMS switching (Quadrupole Time of Flight, MassLynx V4.1 from Waters Corp., USA with the PeptideAuto MFC application V4.0.6.0 from Micromas Ltd.). Calibration of the TOF was done using [Glu¹]-fibrinopeptide B (monoisotopic mass: 1569.67; amino acid sequence: EGVNDNEEGFFSAR. The calibration utilized fragmented masses of this peptide ([M+2H]²⁺, m/z 785.8426, z=2). The MS survey scan was acquired on the range m/z 300-1500 Da with a scan time of 0.9 s and an interscan delay of 0.1 s. The maximum number of ions selected for MSMS was 3 and m/z 50 to 2000 with a scan time of 0.9 s and an interscan delay of 0.1 s was used for MS/MS acquisition. Bovine serum albumin was digested together with samples as a control of method and instrumentation. A specific peak (777.8 Da) was closely monitored and no mass errors and chromatographic malfunctioning were discovered. The mass spectra of fragmented peptides were smoothed (Savitzky Golay), centered, and combined in a merge file. All the MSMS spectra were used to search the UniProt database (16 Nov. 2011, 23224 reviewed sequences/Homo sapiens, reversed decoy sequences were added) with in-house Mascot 2.3.0. Searches were performed with a tolerance on mass measurement of 0.3 Da. The Mascot results were analysed with PeptideShaker ver. 0.12.2. Proteins and peptides were identified with 1% FDR. The false discovery rate (FDR), was calculated as the percentage of positive hits in the decoy database versus the target database both for proteins and peptides. This resulted in 1836 peptides and 416 proteins. Proteins of interest identified with one peptide only were manually verified. LC-MSMS analysis was done at PROBE Laboratory, Proteomic Unit at the University of Bergen, Norway. The bioinformatics tool ProteinCenter 3.8 (Thermo Fisher Scientific, Odense, Denmark) was used to analyze the results of this proteomics study.

Microvesicle Immunoisolation with EpCAM-Beads

M450 Dynabeads coated with sheep anti-mouse antibodies were coated with the anti-EpCAM antibodies as previously described (Flatmark, et al., (2002) Clin. Cancer Res. 8, 444-449). 4 μg of microvesicles were incubated with 10 million EpCAM-dynabeads overnight at 4° C. with rotation. Bead-bound microvesicles were isolated on a magnet and EpCAM-positive microvesicles bound to the beads were eluted by boiling in SDS-sample buffer.

### Western Blot

Normally, 1-3 μg of vesicles were loaded on SDS-PAGE gels and compared to equal amounts of lysates. In some occasions higher amounts of lysates were required to detect specific proteins by Western blot. Pelleted microvesicles pellets directly solubilised in loading buffer. SDS-PAGE gels were transferred to Immobilon-P membranes and then the membranes were blocked with 5% nonfat dry milk and 0.1% Tween 20 in PBS and incubated with the indicated primary antibody. The membranes were then washed three times for at least 5 min with 0.1% Tween 20 in phosphate-buffered saline, and then incubated with secondary antibodies coupled to horseradish peroxidase. Finally, the membranes were washed three times for at least 5 min and developed using an enhanced chemiluminescence detection kit.

### Results

### Microvesicles Released by PC-3 Cells

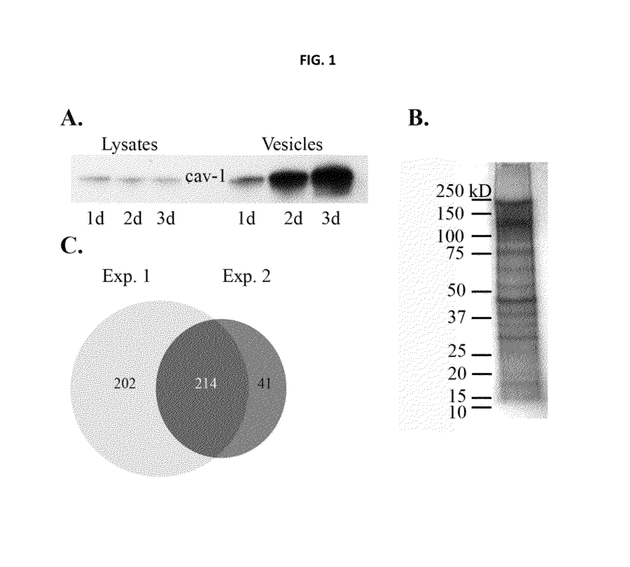

PC-3 cells release a vesicle population that is pelleted from conditioned medium at 100,000 g. These vesicles have previously been collected from serum-free culture medium after 16-24 h (Llorente, et al., (2004) J. Cell Sci. 117, 5343-5351; Llorente, et al., (2007) Eur. J. Cell Biol. 86, 405-415). Since high amounts of microvesicle proteins are required for proteomic analysis (25-50 μg), an experiment was performed to investigate whether longer collection times would result in higher amounts of microvesicle released proteins. Collection of vesicles was started 1, 2 or 3 days after plating and continued for 3, 2 and 1 day respectively. Similar number of cells were collected at the end of the experiment and a trypan blue exclusion test showed that the cells were viable. The release of microvesicles was measured by quantifying the amount of caveolin-1, a protein that is known to be present in these vesicles (Llorente, et al., (2004) J. Cell Sci. 117, 5343-5351) and by measuring the total amount of protein in the vesicles. Both the amount of caveolin-1 (FIG. 1A) and the total amount of protein (data not shown) in the vesicles increased when the collection time was increased. Control experiments showed that the amount of caveolin-1 in cell lysates was similar in the three conditions (FIG. 1A).

### Nano LC-MS/MS Proteomic Analysis of PC-3 Cell-Released Microvesicles

In order to determine the protein composition of vesicles released to the extracellular environment by PC-3 cells, a proteomic analysis using nanocapillary liquid chromatography-tandem mass spectrometry (nano-LC-MS/MS) was performed. Proteins present in the microvesicles were separated by SDS-PAGE using 4-20% gels (FIG. 1B) that were then sliced. Proteins were digested in-gel with porcine trypsin to obtain peptides that were fragmented using nano-LC-MS/MS. The obtained MSMS spectra were used to search the UniProt database (16 Nov. 2011, 23224 reviewed sequences/Homo sapiens) with Mascot 2.3.0. The Mascot results were analysed with PeptideShaker ver. 0.12.2. The database search resulted in the identification of 1836 peptides and 416 proteins with 1% FDR (see Experimental Procedures). Of those proteins, 266 proteins were identified with two or more unique peptide sequences. The annotated spectra of some specific proteins identified with only one peptide that are later mentioned in the manuscript are shown in the supplementary information (FIG. 5).

To determine the reproducibility of the method an independent experiment was performed. Database search resulted in this case in 891 unique peptide sequences and 255 proteins. As shown in FIG. 1C, 214 proteins were found in both experiments thus showing the reproducibility of the method. The fact that a lower number of proteins was found in the second data set may be due to the lower amount of total protein that was analyzed.

### Analysis of Proteins Contained in PC-3 Cell-Released Microvesicles

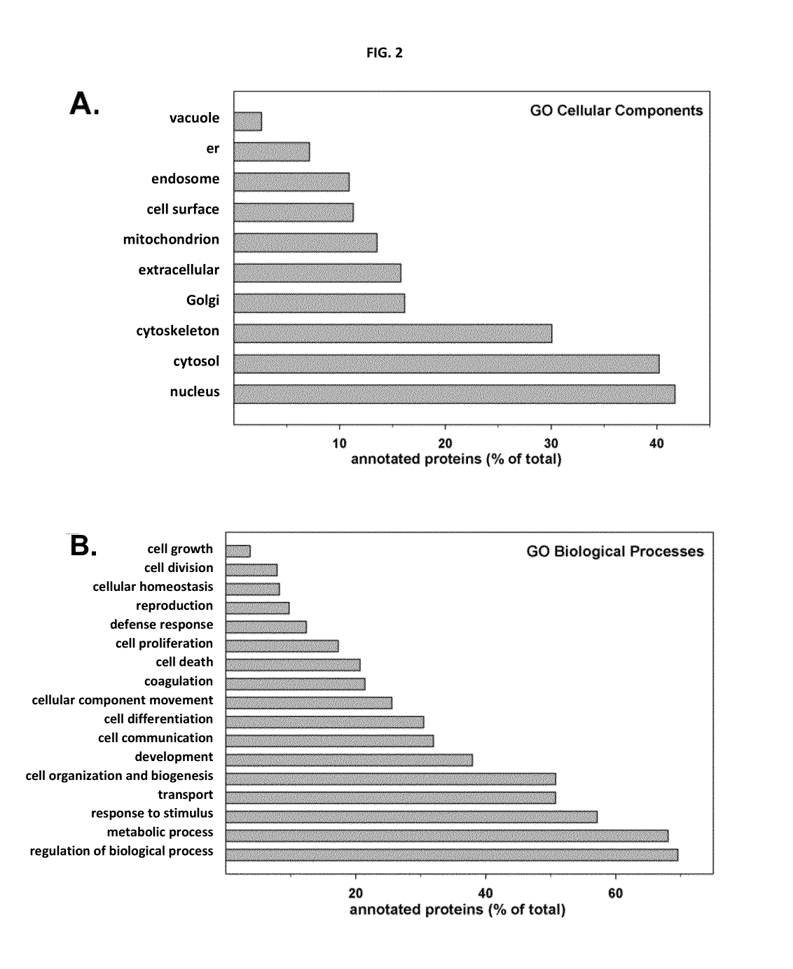

The 266 proteins identified with 2 or more peptide sequences were further analyzed with ProteinCenter 3.8 (Thermo Fisher Scientific, Odense, Denmark), a web-based data interpretation tool that facilitates the comparison and interpretation of data sets. Proteins were classified based on Gene Ontology slim (GOslim) annotations for cellular localization and biological process specifically defined for ProteinCenter. GO annotations often provide several locations and functions for a single protein. From the 266 proteins analyzed, 977 annotations for GOslim cellular components and 1398 annotations for GOslim biological processes were obtained. FIG. 2A shows GOslim anotations for cellular localization. 42 proteins (16%) were annotated as extracellular. Furthermore, only 49 proteins (18%) were predicted to contain signal peptides (PrediSi, PREDIction of SIgnal peptides software) in agreement with the idea that microvesicle proteins are not released by the classical secretory pathway. Furthermore, PC-3 cell-derived microvesicles were not enriched in plasma membrane proteins in agreement with the idea that these vesicles do not originate to a large extent from the plasma membrane. Intracellularly, the proteins were annotated to the cytosol or to different organelles such as nucleus, endosomes or endoplasmic reticulum. A high percentage of proteins were annotated to the category cytoskeleton (FIG. 2A and Table 1). Thus, similarly to other extracellular vesicular populations, proteins found in PC-3 cell microvesicles are intracellularly associated to a variety of cellular organelles (Mathivanan, et al., (2010) Mol. Cell. Proteomics. 9, 197-208).

In FIG. 2B the GOslim annotations for biological processes of the proteins present in PC-3 cell microvesicles are shown. The 5 top annotations for proteins were regulation of biological process, transport (Table 2), metabolic process, response to stimulus and cell organization and biogenesis. Furthermore, pathways annotations were found using the Kyoto Encyclopedia of Genes and Genomes (KEGG) pathway database, an information resource that contain wiring diagrams of molecular interactions and reaction networks (34). Table 3 shows the KEGG pathways to which 13 or more proteins in PC-3 cell microvesicles were annotated. Many proteins in PC-3 cell microvesicles were annotated to the “regulation of actin cytoskeleton” pathway. Furthermore, several proteins were annotated to the “focal adhesion” and the “tight junction” pathways. The annotation “pathways in cancer” and “MAPK signaling pathway”, a pathway that plays a critical role in cancer are also found in Table 3. Finally, 17 proteins were annotated to the KEGG pathway “endocytosis”.

The MS approach used in this proteomic study is not quantitative, but can give an idea of protein abundance based on the number of peptides identifying each protein. In Table 4, the 43 proteins that were identified with highest number of unique peptides (12 or more) are listed. There are several integrins (integrin beta-4, integrin alpha-2, integrin beta-1, integrin alpha-3, integrin alpha-6) and several cytoskeletal associated proteins that facilitate binding of integrins to actin (talin, vinculin, alpha-actinin and filamin) in the list. Integrins are heterodimeric cell surface receptors for extracellular matrix proteins (Barczyk, et al., (2010) Cell Tissue Res. 339, 269-280). Ligand binding causes integrin clustering and recruitment of actin filaments to the integrin cytoplasmic domains. The importance of integrins in cancer is well-known due to their function in cell adhesion, migration, proliferation and cell survival, and prostate cancer is not an exception (Goel, et al., (2009) Am. J. Transl. Res. 1, 211-220). Integrins are commonly found in exosomes where they seem to be involved in anchoring the vesicles to the extracellular matrix (Clayton, et al., (2004) FASEB J. 18, 977-979). Table 4 also contains several transport proteins (e.g., annexin A2, clathrin heavy chain, EH-domain-containing protein 1 and 4), enzymes (e.g., aminopeptidase, pyruvate kinase), heat shock proteins and proteins involved in signal transduction (e.g., 14-3-3 proteins, guanine nucleotide-binding proteins, ephrin type-A receptor 2).

### PC-3 Cell Microvesicles and Exosomes

There are several studies about the protein composition of exosomes, vesicles released after fusion of MVB with the plasma membrane (Simons, M., and Raposo, G. (2009) Curr. Opin. Cell Biol. 21, 575-581). Therefore, the protein composition of PC-3 cells-released microvesicles and exosomes was compared. Exosomes originating from different cell types have a common set and a specific set of proteins. Common proteins found in exosomes are integrins, heat shock proteins, tetraspanins, proteins involved in vesicular transport and cellular signaling and cytoskeletal proteins. Integrins, heat shock proteins and signaling proteins are widely represented in PC-3 cell-released microvesicles. Also several tetraspanin proteins, membrane proteins characterized by the presence of four hydrophobic domains, were found in these vesicles: CD9, CD63, CD81 and CD151. In addition, tetraspanin9 and tetraspanin15 were identified with only one peptide (FIG. 5). Typical cytoskeletal and vesicular transport proteins in PC-3 microvesicles have been listed in Table 1 and Table 2 respectively. Table 2 includes for example clathrin and several members of the Rab, annexin and ARF family of proteins. These proteins are involved in one or several transport mechanism along the endocytic and/or exocytic pathway and are not specific for microvesicle release. However, there are regulators of exosome release among these proteins. In fact, several Rab proteins, Rab11 (Savina, et al., (2002) J. Cell Sci. 115, 2505-2515), Rab27 (Ostrowski, et al., (2010) Nat. Cell Biol. 12, 19-30) and Rab35 (Hsu, et al., (2010) J. Cell Biol. 189, 223-232), have already been shown to regulate exosome release. EH-domain-containing proteins 1 and 4 were identified with a high number of peptides (Table 4). These proteins belong to the EPS 15 homology (EH) domain-containing proteins family and have previously been involved in early endosomal transport and recycling (Caplan, et al.,. (2002) EMBO J. 21, 2557-2567; Sharma, et al., (2008) Traffic. 9, 995-1018). Moreover, myoferlin, a protein recently implicated in the endocytic pathway (Bernatchez, et al., (2009) Am. J. Physiol Cell Physiol. 297, C484-C492) that binds to EH domain-containing family members (Doherty, et al., (2008) J. Biol. Chem. 283, 20252-20260) is also present in PC-3 microvesicles.

Since the vesicles released by PC-3 cells may proceed from MVB, the presence in PC-3 cell-derived microvesicles of proteins normally located in MVB/lysosomes was then investigated. In fact two MVB associated proteins, CD63 and the endosomal sorting complex required for transport (ESCRT) protein Alix (programmed cell death 6-interacting protein), are commonly found in exosomes and have been considered as exosome markers (Simons, et al., (2009) Curr. Opin. Cell Biol. 21, 575-581). Both CD63 and Alix are found in PC-3 microvesicles. Furthermore, several ESCRT (vps28, CHMP1B, CHMP4B) were identified with only one peptide (FIG. 5).

The proteins identified in PC-3 cell microvesicles proteins where then compared to the proteins found in Exocarta 3.2, a compendium for proteins identified in exosomes (Mathivanan, S., and Simpson, R. J. (2009) Proteomics. 9, 4997-5000). First, the presence in PC-3 cell microvesicles of the top 25 proteins often identified in exosomes was investigated. Only two proteins (major histocompatibility complex class II and syntenin) were not found in PC-3 cell-derived microvesicles. Moreover, only 6% of the proteins in PC-3 cell microvesicles were not found in Exocarta. However, this number may be smaller since these proteins may have been identified in studies not included in Exocarta. It should also be mentioned that several of these proteins belong to families of proteins that are described in Exocarta. In conclusion, the fact that most of the proteins in PC-3 cell microvesicles, including several MVB proteins considered as exosomal markers, have previously been identified in exosomes is in agreement with the idea that microvesicles released from PC-3 cells are exosomes released by prostate cells.

### Identification of Cancer Relevant Proteins in PC-3 Cell Microvesicles

It was next investigated whether there were proteins in PC-3 microvesicles associated with prostate cancer. In particular, three proteins were considered especially relevant: the membrane glycoprotein CUB domain-containing protein 1 protein (CDCP1), the tetraspanin CD151 and CD147 (also named basigin).

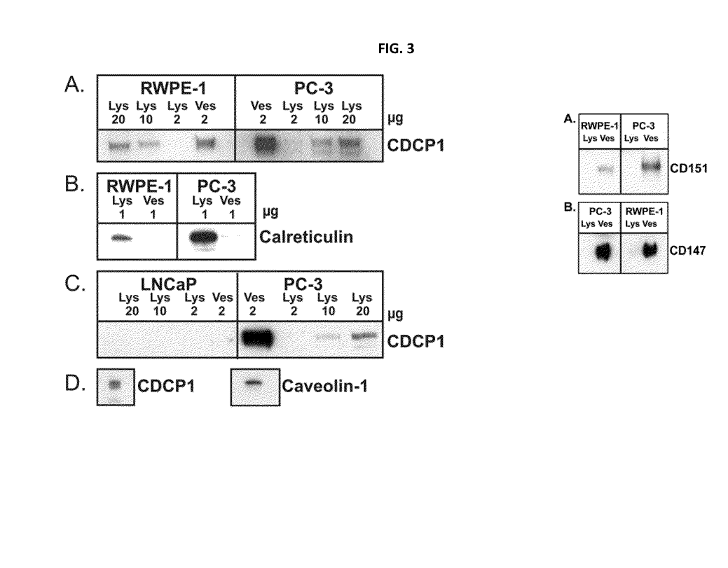

CDCP1 was investigated first since this protein was not found in Exocarta, and is thus more specific of prostate cancer released microvesicles. CDCP1 has been described as a tumor marker (Wortmann, et al., (2009) IUBMB. Life. 61, 723-730), and it has been proposed to function as an antiapoptotic molecule that facilitates tumor cell survival during metastasis (Deryugina, et al., (2009) Mol. Cancer. Res. 7, 1197-1211). Furthermore, it has been shown that a monoclonal antibody against CDCP1 inhibits metastasis in a prostate cancer model (Siva, et al., (2008) Cancer Res. 68, 3759-3766). As shown in FIG. 3A, the presence of this protein in microvesicles was confirmed by Western blot. The CDCP1 band appears at a molecular weight (between the molecular markers 100 and 150 kDa) that corresponds to the glycosylated form of the protein. Furthermore, when equal amounts (2 μg) of protein from microvesicles and cell lysates were analyzed by SDS-PAGE and Western blot, CDCP1 was only weakly detected in the lysates (FIG. 3A), thus showing that CDCP1 is enriched in microvesicles. Higher amounts of lysates (10 μg) were required to better detect CDCP1 in PC-3 lysates (FIG. 3A). As a control, the presence of the endoplasmic reticulum protein calreticulin was investigated. This protein was mainly detected in the lysates of both PC-3 and RWPE-1 cells and not significantly in the released microvesicles (FIG. 3B). In order to investigate whether normal prostate epithelial cells release vesicles containing CDCP1, the RWPE-1 cell line was used. As shown in FIG. 3A, the amount of CDCP1 was significantly lower in RWPE-1 cell-released microvesicles than in microvesicles released from PC-3 cells when equal amounts of vesicles were loaded. Furthermore, when the levels of CDCP1 in microvesicles released from the nonmetastatic prostate cancer cell line LNCaP and from PC-3 were compared, again the amount of CDCP1 was significantly higher in microvesicles released by PC-3 cells (FIG. 3C). Based on these results, the detection of CDCP1 in microvesicles isolated from biological fluids e.g., urine, serum, prostatic fluid, seminal fluid, finds use for detection of metastatic prostate cancer. Isolation of microvesicles released from epithelial cells from biological fluids can be performed with beads coupled to an antibody to epithelial cell adhesion molecule (EpCAM) (Taylor, D. D., and Gercel-Taylor, C. (2008) Gynecol. Oncol. 110, 13-21). This molecule is expressed exclusively in epithelia and in epithelial-derived cancer types, and increased levels of EpCAM have been associated with the early development of prostatic adenocarcinoma (Poczatek, et al., (1999) J. Urol. 162, 1462-1466). Since the MS analysis showed that EpCAM is found in microvesicles released by the epithelial prostate cancer line PC-3, it was investigated whether it was possible to immunoisolate PC-3 cell microvesicles and measure CDCP-1 protein levels. As shown in FIG. 3D, EpCAM conjugated dynabeads were able to immunoisolate PC-3 released vesicles containing CDCP1. The presence of caveolin-1 in the immunoisolated microvesicles was also determined to confirm the binding of microvesicles to the beads (FIG. 3D).

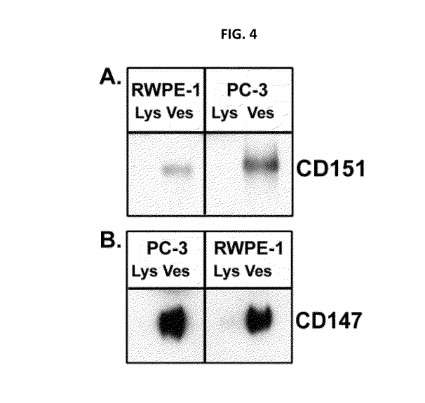

The presence of the tetraspanin CD151 in PC-3 cells-released exosomes was also verified by Western blot. This protein has been shown to be overexpressed in several cancers, and it induces tumor progression by regulating cell migration through its association with integrins and matrix metalloproteinases (Zoller, M. (2009) Nat. Rev. Cancer. 9, 40-55). Furthermore, it has been reported that CD 151 protein expression predicted the clinical outcome of low-grade primary prostate cancer better than histologic grading (Ang, et al., (2004) Cancer Epidemiol. Biomarkers Prev. 13, 1717-1721). As shown in FIG. 4A, this protein was enriched in PC-3 cell-released microvesicles compared to lysates. Furthermore, similarly to CDCP1, the levels of this protein were higher in PC-3 cells released microvesicles than in microvesicles released from RWPE-1 cells.

In conclusion, CDCP1 and CD151 are useful prostate cancer biomarkers since both CDCP1 and CD151 are enriched in vesicles released by prostate cancer cells compared to vesicles releases by normal epithelial prostate cells, and it has been shown that cancer cells release more vesicles than normal cells (Ang, et al., (2004) Cancer Epidemiol. Biomarkers Prev. 13, 1717-1721). In fact, one of these molecules, CD151, has already been shown to be useful as a prostate cancer biomarker in prostate cancer tissue (Ang, et al., (2004) Cancer Epidemiol. Biomarkers Prev. 13, 1717-1721). Detection of biomarkers in microvesicles has the advantage of being less invasive. In addition, the general problem of tumor heterogeneity can be avoided.

The third candidate, CD147, also was validated by SDS-PAGE and Western Blot. It has recently been described that this protein has a role as an independent predictor of biochemical recurrence, development of metastasis and reduced overall survival in prostate cancer (Zhong, et al., (2012) Int. J. Cancer. 130, 300-308). As shown in FIG. 4B, when equal amounts of protein from PC-3 cell microvesicles and cell lysates were analyzed by SDS-PAGE and Western blot, CD147 was clearly enriched in microvesicles released from PC-3 cells. The levels of CD147 in microvesicles from the normal epithelial cell line were not very different from the levels found in microvesicles released by PC-3 cells.

Finally, other proteins found in PC-3 microvesicles that are known to be associated to and/or disregulated in prostate cancer are translationally controlled tumor protein (TCTP) and neuropilin. TCTP is an anti apoptotic protein highly expressed in prostate cancer (Gnanasekar, et al, (2009) Int. J. Oncol. 34, 1241-1246) and neuropilin, a receptor for vascular endothelial growth factor (VEGF), has been associated with tumor angiogenesis and migration (Miao, et al., (2000) FASEB J. 14, 2532-2539; Jia, et al., (2010) Br. J. Cancer. 102, 541-552), and it has also been described as a marker for prostate cancer aggressiveness (Latil, et al., (2000) Int. J. Cancer. 89, 167-171). Vinculin, an actin binding protein involved in the interaction between the actin cytoskeleton and the extracellular matrix, was very abundant in PC-3 microvesicles (Table 4). It has recently been reported that vinculin expression is associated with increased tumor cell proliferation and progression in advanced prostate cancer (Ruiz, et al., (2011) J. Pathol. 223, 543-552). Many other proteins found in PC-3 microvesicles are known to be involved in several cancer diseases (e.g., 1pha-enolase, catenins, ephrin type-A receptor, epidermal growth factor receptor, several proteins of the Ras family and several CD proteins such as CD4, aminopeptidase-N (CD13), CD81).

Comparison Between the Protein Composition of PC-3 Cell Microvesicles and Microvesicles Derived from Other Cancer Cells

During the last years several proteomic studies of microvesicles isolated from cancer cell lines have been performed. It was investigate to which extent the proteins identified in PC-3 cell microvesicles overlap with the proteins present in microvesicles released by other cancer cells. A recent study identified 48 proteins with two or more peptides in vesicles released by the human prostate cancer cell line PC346C (Jansen, et al., (2009) Mol. Cell. Proteomics. 8, 1192-1205). Rab proteins, Arf proteins, Rho proteins and guanine nucleotide binding proteins for example were not found in this study and only one integrin family member and one member of the 14-3-3 family of proteins were found. Furthermore, a proteomic analysis of vesicles derived from prostate-cancer metastasis identified 30 proteins (Ronquist, et al., (2010) Anticancer Res. 30, 285-290), only 11 of them were identified in PC-3 cell microvesicles. Annexin isoforms, alpha-enolase, 14-3-3 protein sigma, actin, peroxiredoxin-6, ubiquitin-conjugating enzyme E2N, triosephosphate isomerase, phosphatidylethanolamide-binding protein and heat shock protein beta-1 were found in the two vesicle populations. Also in that study important components of microvesicles from PC-3 cells such as integrins, Rab proteins or tetraspanins were not found. A list of proteins from microvesicles released from PC-3 cells is described (Inder, et al., (2011) Mol. Cell. Proteomics. Epub ahead of print, Manuscript M111.012245).

Finally, the overlap between the 266 proteins identified in PC-3 microvesicles and the 353 proteins identified in vesicles released by a bladder cancer cell line (Welton, et al., (2010) Mol. Cell. Proteomics. 9, 1324-1338) and the 394 proteins identified in vesicles released by a colon cancer cell line (Mathivanan, et al., (2010) Mol. Cell. Proteomics. 9, 197-208) was investigated. Approximately 35% of the proteins in PC-3 cell-derived microvesicles were found in bladder cancer cells and in colon cancer microvesicles. In general, these vesicle populations did not overlap to a high extent.

TABLE 1
Cytoskeleton related proteins present in PC-3 cells microvesicles.
Proteins of the Ras superfamily are not included.

| Protein name | Acc. no |
|---|---|
| Actin, aortic smooth muscle | P62736 |
| Actin, cytoplasmic 1 | P60709 |
| Actin-related protein 2 | P61160 |
| Actin-related protein 2/3 complex subunit 2 | O15144 |
| Actin-related protein 2/3 complex subunit 3 | O15145 |
| Actin-related protein 2/3 complex subunit 4 | P59998 |
| Actin-related protein 2/3 complex subunit 5- | Q9BPX5 |
| like protein | |
| Alpha-actinin-1 | P12814 |
| Alpha-actinin-4 | O43707 |
| Catenin beta-1 | P35222 |
| Catenin delta-1 | O60716 |
| Coactosin-like protein | Q14019 |
| Cofilin-1 | P23528 |
| Destrin | P60981 |
| Dynein heavy chain | Q14204 |
| Ezrin | P15311 |
| F-actin-capping protein subunit alpha-1 | P52907 |
| Fascin | Q16658 |
| Filamin-A | P21333 |
| Filamin-B | O75369 |
| Filamin-C | Q14315 |
| Junction plakoglobin | P14923 |
| Moesin | P26038 |
| Myosin Ib | O43795 |
| Myosin Ic | O00159 |
| Myosin Id | O94832 |
| Myosin-9 | P35579 |
| Myristoylated alanine-rich C-kinase substrate | P29966 |
| Nck-associated protein 1 | Q9Y2A7 |
| Plastin-2 | P13796 |
| Plastin-3 | P13797 |
| Plectin-1 | Q15149 |
| Plexin-B2 | O15031 |
| Profilin-1 | P07737 |
| Radixin | P35241 |
| Septin-2 | Q15019 |
| Talin-1 | Q9Y490 |
| Tropomyosin alpha-3 chain | P06753 |
| Tubulin alpha-1B chain | P68363 |
| Tubulin beta chain | P07437 |
| Tubulin beta-4 chain | P04350 |
| Vinculin | P18206 |

TABLE 2
Proteins involved in vesicular transport present in
PC-3 cells microvesicles.

| Protein name | Acc. No. |
|---|---|
| ADP-ribosylation factor 3 | P61204 |
| ADP-ribosylation factor 4 | P18085 |
| ADP-ribosylation factor 5 | P84085 |
| ADP-ribosylation factor 6 | P62330 |
| Alpha-soluble NSF attachment protein | P54920 |
| Annexin A1 | P04083 |
| Annexin A2 | P07355 |
| Annexin A3 | P12429 |
| Annexin A5 | P08758 |
| Annexin A6 | P08133 |
| Annexin A7 | P20073 |
| Annexin A11 | P50995 |
| Clathrin heavy chain 1 | Q00610 |
| EH-domain-containing protein 1 | Q9H4M9 |
| EH-domain-containing protein 4 | Q9H223 |
| Heat shock 70 kDa protein 1A/1B | P08107 |
| Myoferlin | Q9NZM1 |
| Programmed cell death 6-interacting protein | Q8WUM4 |
| Rab-1A | P62820 |
| Rab-1B | Q9H0U4 |
| Rab-5A | P20339 |
| Rab-5B | P61020 |
| Rab-7a | P51149 |
| Rab-8A | P61006 |
| Rab-8B | Q92930 |
| Rab-10 | P61026 |
| Rab-11A | P62491 |
| Rab-12 | Q61Q22 |
| Rab-13 | P51153 |
| Rab-14 | P61106 |
| Rab-22A | Q9UL26 |
| Rab-27B | O00194 |
| Rab-35 | Q15286 |
| Syntaxin-4 | Q12846 |
| Syntaxin-binding protein 3 | O00186 |
| Vesicle-associated membrane protein 8 | Q9BV40 |

TABLE 3
KEGG* pathways to which 13 or more proteins in
PC-3 cells microvesicles were annotated.

| KEGG ID | KEGG Pathway | No. proteins |
|---|---|---|
| hsa04810 | Regulation of actin cytoskeleton | 30 |
| hsa04510 | Focal adhesion | 22 |
| hsa04670 | Leukocyte transendothelial migration | 18 |
| hsa04144 | Endocytosis | 17 |
| hsa05200 | Pathways in cancer | 16 |
| hsa04530 | Tight junction | 16 |
| hsa04010 | MAPK signaling pathway | 15 |
| hsa04722 | Neurotrophin signaling pathway | 15 |
| hsa04062 | Chemokine signaling pathway | 14 |
| hsa05130 | Pathogenic Escherichia coli infection | 14 |
| hsa04145 | Phagosome | 13 |
| hsa04360 | Axon guidance | 13 |
| hsa05100 | Bacterial invasion of epithelial cells | 13 |

*KEGG: Kyoto Encyclopedia of Genes and Genomes

TABLE 4
Proteins identified with 12 or more unique peptides
in PC-3 cells microvesicles.

| Unique | Accession | Protein |
|---|---|---|
| peptides | number | Name |
| 39 | P16144 | Integrin beta-4 (CD104 antigen) |
| 33 | P18206 | Vinculin |
| 31 | P15144 | Aminopeptidase N (CD13) |
| 30 | P12814 | Alpha-actinin-1 |
| 28 | P17301 | Integrin alpha-2 (CD49b antigen) |
| 27 | P05556 | Integrin beta-1 (CD29 antigen) |
| 25 | P26006 | Integrin alpha-3 (CD49c antigen) |
| 24 | P21333 | Filamin-A |
| 24 | P26038 | Moesin |
| 23 | O75369 | Filamin-B |
| 23 | P23229 | Integrin alpha-6 (CD49f antigen) |
| 22 | O60716 | Catenin delta-1 |
| 22 | P05023 | Sodium/potassium-transporting ATPase |
| | | subunit alpha-1 |
| 22 | P07355 | Annexin A2 |
| 22 | Q15149 | Plectin |
| 19 | Q9H4M9 | EH domain-containing protein 1 |
| 18 | P60709 | Actin, cytoplasmic 1 |
| 18 | O00159 | Myosin-Ic |
| 18 | O43854 | EGF-like repeat and discoidin I-like |
| | | domain-containing protein 3 |
| 18 | P11142 | Heat shock cognate 71 kDa protein |
| 18 | P29317 | Ephrin type-A receptor 2 |
| 16 | P62736 | Actin, aortic smooth muscle |
| 16 | O43707 | Alpha-actinin-4 |
| 16 | P14923 | Junction plakoglobin |
| 16 | P63104 | 14-3-3 protein zeta/delta |
| 15 | P27348 | 14-3-3 protein theta |
| 15 | P35222 | Catenin beta-1 |
| 15 | Q09666 | Neuroblast differentiation-associated |
| | | protein AHNAK |
| 14 | O43795 | Myosin-Ib |
| 14 | P07900 | Heat shock protein HSP 90-alpha |
| 14 | P14618 | Pyruvate kinase isozymes M1/M2 |
| 14 | P31946 | 14-3-3 protein beta/alpha |
| 13 | P04899 | Guanine nucleotide-binding protein |
| | | G(i) subunit alpha-2 |
| 13 | P08238 | Heat shock protein HSP 90-beta |
| 13 | P15311 | Ezrin |
| 13 | P62937 | Peptidyl-prolyl cis-trans isomerase A |
| 13 | Q7L576 | Cytoplasmic FMR1-interacting protein 1 |
| 13 | Q9H223 | EH domain-containing protein 4 |
| 12 | P35241 | Radixin |
| 12 | P62258 | 14-3-3 protein epsilon |
| 12 | Q00610 | Clathrin heavy chain 1 |
| 12 | Q9H5V8 | CUB domain-containing protein 1 |
| 12 | Q9P265 | Disco-interacting protein 2 homolog B |

### Example 2

Proteins identified in microvesicles derived from PC3 cells were analyzed in microvesicles from urine of two control men and one prostate cancer patient (collected just before prostatectomy). A method based on ultracentrifugation (see methods) was used to isolate microvesicles from urine. Their protein composition was subsequently analyzed by mass spectrometry. The two control samples were grouped and compared to the prostate cancer sample. From the proteins identified in the prostate cancer urinary microvesicles, 157 proteins were common with the proteins identified in PC-3 derived microvesicles (FIG. 5). They have been classified based on fold upregulation or down-regulation in the prostate cancer urinary microvesicles compared to control urinary microvesicles. Twenty two of these proteins were enriched more than 1.5 times in the prostate cancer urinary microvesicles, while 13 were down-regulated by at least 2-fold. These diagnostically relevant proteins are summarized in Table 5.

### Methods

### Urinary Microvesicles Isolation

Urine was collected from control men or prostate cancer patients and processed within 2-3 hours. The collection of urine was approved by the Regional Committe for Medical and Health Research Ethics (2012/335 C). Urinary microvesicles were isolated using sequential centrifugation and pelleted at 100,000 g in a similar way as PC-3 derived microvesicles (Sandvig K, Llorente A. Mol Cell Proteomics. 2012.11(7):M111.012914).

### Protein Determination

The protein content of urinary vesicles was determined using a bicinchoninic acid protein assay kit according to the manufacturer's instructions.

### In-Solution Digestion of Exosomes

To identify proteins in urinary microvesicles, one volume of vesicles in solution was added to four volumes of cold acetone (with 1M HCl) and methanol at −20° C. After mixing, the sample was centrifuged at 15,000×g for 15 min. The supernatant was aspirated and the pellet was dried in Speed-Vac. The pellet was then dissolved in 50 μl fresh 100 mM ammonium bicarbonate with 6 M urea, and sequentially reduced with 10 mM dithiothreitol at 30° C. for 30 min. Samples were then incubated with 25 mM iodoacetamide to alkylate exposed side chains for 1 h at room temperature away from light. The enzymatic digestion was initiated by adding 1 μg Lys-C and incubating at 37° C. for 2 h. 240 μl 50 mM ammonium bicarbonate with 10 μg trypsin was added and first incubated 1 h at 37° C., followed by 15 h at 30° C. Prior LC-MS analysis, 5 μl formic acid was added to the digested vesicles.

### Nano LC-MS/MS

The dried peptides were dissolved in 10 μl 1% formic acid, 5% MeCN in water and half the volume was injected into an Ultimate 3000 nanoLC system (Dionex, Sunnyvale Calif., USA) connected to a linear quadrupole ion trap-orbitrap (LTQ-Orbitrap XL) mass spectrometer (ThermoScientific, Bremen, Germany) equipped with a nanoelectrospray ion source. An Acclaim PepMap 100 column (C18, 3 μm, 100 Å) (Dionex) with a capillary of 25 cm bed length was used for separation by liquid chromatography. A flow rate of 300 mL/min was employed with a solvent gradient of 7% B to 50% B in 110 min. Solvent A was 0.1% formic acid, whereas aqueous 90% acetonitrile in 0.1% formic acid was used as solvent B.

The mass spectrometer was operated in the data-dependent mode to automatically switch between Orbitrap-MS and LTQ-MS/MS acquisition. Survey full scan MS spectra (from m/z 300 to 2000) were acquired in the Orbitrap with resolution R=60,000 at m/z 400 (after accumulation to a target of 500,000 charges in the LTQ). The method used allowed sequential isolation of the most intense ions, up to six, depending on signal intensity, for fragmentation on the linear ion trap using collisional induced dissociation (CID) at a target value of 10,000 charges. For accurate mass measurements the lock mass option was enabled in MS mode and the polydimethylcyclosiloxane (PCM) ions generated in the electrospray process from ambient air were used for internal recalibration during the analysis.

### Data Analysis

Raw LTQ Orbitrap XL data were analyzed with MaxQuant (Cox & Mann 2008 Nat Biotech) (v.1.2.2.5) utilizing the Andromeda search engine. The database search was performed by database comparisons with human entries (87,061 sequences) from IPI (v. 3.68) (Perkins et al 1999 Electrophoresis). Trypsin was selected as enzyme allowing one missed cleavage site. Tolerance of 10 ppm for the precursor ion and 0.5 Da for the MS/MS fragments was applied. Methionine oxidation, acetylation at protein N-terminus, deamidation of glutamines and asparagines and were allowed as variable modifications. In MaxQuant, reversed sequences and common contaminants were included in the database search enabling estimation of the false discovery rate (FDR). This was set to 0.1% for protein and peptide identifications. The false discovery rate (FDR), was calculated as the percentage of positive hits in the decoy database versus the target database both for proteins and peptides. The bioinformatics tool Scaffold4 (ver 4.3.0) was used to analyze the results of this study.

### Results

TABLE 5
Common proteins between microvesicles derived from
the prostate cancer cell line PC-3 and urinary
microvesicles from prostate cancer that are
differentially expressed.

| | |
|---|---|
| | Accesion |
| Protein name | number |

Increased at least 2-fold

| | |
|---|---|
| Ephrin-B1 | P98172 |
| Integrin alpha-3 | P26006 |
| Integrin alpha-V | P06756 |
| Lactadherin | Q08431 |
| 5′-nucleotidase | P21589 |
| CD63 antigen | P08962 |
| Septin-2 | Q15019 |
| Puromycin-sensitive aminopeptidase | P55786 |

Increased at least 1.5-fold

| | |
|---|---|
| CD81 antigen | P60033 |
| Chloride intracellular channel protein 4 | Q9Y696 |
| Myristoylated alanine-rich C-kinase substrate | P29966 |
| L-lactate dehydrogenase A chain | P00338 |
| Annexin A6 | P08133 |
| Cytoplasmic FMR1-interacting protein 1 | Q7L576 |
| Peroxiredoxin-5, mitochondrial | P30044 |
| Ras-related protein Rab-14 | P61106 |
| Protein NDRG1 | Q92597 |
| Rho-related GTP-binding protein RhoF | Q9HBH0 |
| DnaJ homolog subfamily C member 5 | Q9H3Z4 |
| Integrin beta-1 | P05556 |
| Peptidyl-prolyl cis-trans isomerase FKBP1A | P62942 |
| Protein FAM49B | Q9NUQ9 |

Reduced at least 2-fold

| | |
|---|---|
| Basigin | P35613 |
| Actin-related protein 2/3 complex subunit 2 | O15144 |
| F-actin-capping protein subunit alpha-1 | P52907 |
| Proto-oncogene tyrosine-protein kinase Src | P12931 |
| GTPase NRas | P01111 |
| Ras-related protein Rap-2c | Q9Y3L5 |
| Ras-related protein Rap-2b | P61225 |
| Protein XRP2 | O75695 |
| Ras-related protein Rab-22A | Q9UL26 |
| Protein S100-A14 | Q9HCY8 |
| Fatty acid synthase | P49327 |
| Long-chain-fatty-acid--CoA ligase 4 | O60488 |
| Mucin-5B | Q9HC84 |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of identifying prostate cancer in a sample from a subject, comprising:
(a) detecting the presence, absence, or altered level of at least one polypeptide selected from the group consisting of CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, and mucin-5B with a reagent that specifically detects said polypeptide in a sample from a subject, wherein said sample comprises a microvesicle; and
(b) identifying the presence of prostate cancer in the sample when said at least one polypeptide is present in the sample.

2. The method of claim 1, wherein the sample is selected from the group consisting of tissue, blood, plasma, serum, urine, urine supernatant, urine cell pellet, semen, prostatic secretions and prostate cells.

3. The method of claim 1, wherein detection is carried out utilizing a method selected from the group consisting of a spectrometry technique, a chromatography technique, and an immunoassay.

4. The method of claim 1, wherein said reagent is an antibody that specifically binds to said polypeptide.

5. The method of claim 1, wherein said method further comprises the step of enriching said sample for the presence of said microvesicle.

6. The method of claim 5, wherein said method further comprises the step of isolating said microvesicle from a said sample.

7. The method of claim 6, wherein said microvesicles are isolated from said sample by antibody capture.

8. The method of claim 7, wherein said antibody capture utilizes a bead comprising an antibody that specifically binds to said microvesicle.

9. The method of claim 8, wherein said antibody specifically binds to epithelial cell adhesion molecule (EpCAM).

10. The method of claim 1, wherein said microvesicle is released from an epithelial cell.

11. The method of claim 10, wherein said epithelial cell is a prostate cancer cell.

12. The method of claim 11, wherein said prostate cancer cell is a metastatic prostate cancer cell.

13. The method of claim 1, wherein said detecting further comprises detecting the presence of a complex of said polypeptide and said reagent.

14. The method of claim 1, further comprising the step of treating said subject for prostate cancer when said polypeptide is detected.

15. A kit, comprising reagents for detection of the presence, absence, or level of at least two polypeptides selected from the group consisting of CUB domain-containing protein 1 protein (CDCP1), CD151, CD147, translationally controlled tumor protein (TCTP), neuropilin, ephrin-B1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, and mucin-5B.

16. The kit of claim 1, wherein said reagent is an antibody that specifically binds to said polypeptide.

17. The kit of claim 15, wherein said kit comprises reagents for detecting at least three of said polypeptides.

18. A complex, comprising: at least two polypeptides selected from the group consisting of CUB domain-containing protein 1 protein (CDCP1), CD151, CD147, translationally controlled tumor protein (TCTP), neuropilin, ephrin-B 1, integrin alpha-3, integrin alpha-V, lactadherin, 5′-nucleotidase, CD63 antigen, septin-2, puromycin-sensitive aminopeptidase, CD81 antigen, chloride intracellular channel protein 4, myristoylated alanine-rich C-kinase substrate, L-lactate dehydrogenase A chain, annexin A6, cytoplasmic FMR1-interacting protein 1, mitochondrial Peroxiredoxin-5, Ras-related protein Rab-14, protein NDRG1, Rho-related GTP-binding protein RhoF, DnaJ homolog subfamily C member 5, integrin beta-1, peptidyl-prolyl cis-trans isomerase FKBP1A, protein FAM49B, basigin, actin-related protein ⅔ complex subunit 2, F-actin-capping protein subunit alpha-1, proto-oncogene tyrosine-protein kinase Src, GTPase NRas, Ras-related protein Rap-2c, Ras-related protein Rap-2b, protein XRP2, Ras-related protein Rab-22A, protein S100-A14, fatty acid synthase, long-chain-fatty-acid—CoA ligase 4, and mucin-5B, each of which is complexed to a reagent that specifically detects said polypeptide.

19. The complex of claim 18, wherein said reagent is an antibody that specifically binds to said polypeptide.

20. The complex of claim 18, wherein said complex comprises complexes of at least three of said polypeptides.


SEQUENCE LISTING (1 sequence)

SEQ ID NO: 1 - protein, 14 aa, Artificial Sequence [sequence omitted]